(19) United States
(12) Patent Application Publication
(10) Pub. No.: US 2023/0007082 A1
(43) Pub. Date: Jan. 5, 2023

(54) POLLUTANT SENSOR PLACEMENT

(71) Applicant: Microsoft Technology Licensing, LLC, Redmond, WA (US)

(72) Inventors: Conor E. KELLY, Dublin (IE); Ashish BHATIA, Bedford, MA (US); Yagna Deepika ORUGANTI, Houston, TX (US); Peeyush KUMAR, Redmond, WA (US); Anirudh BADAM, Issaquah, WA (US); Leonardo DE OLIVEIRA NUNES, Rio de Janeiro (BR); Shirui WANG, Houston, TX (US); Yazeed ALAUDAH, Houston, TX (US); Neera B. TALBERT, Houston, TX (US); Xinyu CHEN, Houston, TX (US); Fatemeh ZAMANIAN, Houston, TX (US)

(73) Assignee: Microsoft Technology Licensing, LLC, Redmond, WA (US)

(21) Appl. No.: 17/363,202

(22) Filed: Jun. 30, 2021

(51) Int. Cl. H04L 67/12; G16Y 40/35; G16Y 20/10; G06N 7/00; G16Y 40/10

(52) U.S. Cl. CPC H04L 67/12; G16Y 40/35; G16Y 20/10; G06N 7/00; G16Y 40/10

(57) ABSTRACT

A method for pollutant sensor placement is described. Data about environmental characteristics across a geographic region is received from a plurality of environmental sensors. The geographic region includes one or more pollutant sources that emit a pollutant. The received data is transformed from one or more of the plurality of environmental sensors into common data having a common grid across the geographic region. The geographic region is divided into a plurality of sub-regions based on the common data. Locations within the geographic region are determined for placement of pollutant sensors based on estimated dispersion of the pollutant through the plurality of sub-regions.

### BACKGROUND

Extensive efforts have been made to improve detection and remediation of atmospheric leaks of methane, which is a potent greenhouse gas. Methane sources that leak into the atmosphere may include oil and gas industry infrastructure (e.g., equipment and industrial sites for production, processing, transmission, storage, and distribution), as well as agricultural sites, landfills, and abandoned oil and gas fields. There are a variety of methane-sensing technologies being deployed, with varying sensitivities, spatial and temporal resolution, and price of acquisition. These technologies include satellites, aerial surveys, Internet of Things (IoT) sensor grids, unmanned vehicles, and other sensors. Satellite data, for example, from Sentinel-5P TROPOMI may have coarse spatial resolution and low methane detection sensitivity but near-daily global coverage. Aircraft surveys, on the other hand, may provide a much richer picture of an area of interest with higher spatial resolution, but such surveys are intermittent. Ground sensors may provide real-time data streams, but may have a high cost associated with deploying sensor grids to cover large swaths of land.

It is with respect to these and other general considerations that aspects have been described. Also, although relatively specific problems have been discussed, it should be understood that the aspects should not be limited to solving the specific problems identified in the background.

### SUMMARY

Aspects of the present disclosure are directed to placement of sensors for pollutant detection.

In one aspect, a method for pollutant sensor placement is provided. Data about environmental characteristics across a geographic region is received from a plurality of environmental sensors. The geographic region includes one or more pollutant sources that emit a pollutant. The received data is transformed from one or more of the plurality of environmental sensors into common data having a common grid across the geographic region. The geographic region is divided into a plurality of sub-regions based on the common data. Locations within the geographic region are determined for placement of pollutant sensors based on estimated dispersion of the pollutant through the plurality of sub-regions.

In another aspect, a system for pollutant sensor placement is provided. The system includes at least one processor and at least one memory storing computer-executable instructions that when executed by the at least one processor cause the system to: receive data about environmental characteristics across a geographic region from a plurality of environmental sensors, wherein the geographic region includes one or more pollutant sources that emit a pollutant; transforming the received data from one or more of the plurality of environmental sensors into common data having a common grid across the geographic region; divide the geographic region into a plurality of sub-regions based on the common data; and determine locations within the geographic region for placement of pollutant sensors based on estimated dispersion of the pollutant through the plurality of sub-regions.

In yet another aspect, a system for pollutant sensor placement is provided. The system includes a staging database, a sensor data processor, and a deployment processor. The staging database is configured to receive data about environmental characteristics across a geographic region from a plurality of environmental sensors. The geographic region includes one or more pollutant sources that emit a pollutant. The sensor data processor is configured to transform the received data from one or more of the plurality of environmental sensors into common data having a common grid across the geographic region. The deployment processor is configured to: divide the geographic region into a plurality of sub-regions based on the common data; and determine locations within the geographic region for placement of pollutant sensors based on estimated dispersion of the pollutant through the plurality of sub-regions.

This summary is provided to introduce a selection of concepts in a simplified form that are further described below in the Detailed Description. This summary is not intended to identify key features or essential features of the claimed subject matter, nor is it intended to be used to limit the scope of the claimed subject matter.

### BRIEF DESCRIPTION OF THE DRAWINGS/FIGURES

Non-limiting and non-exhaustive examples are described with reference to the following Figures.

FIG. 1 shows a block diagram of an example of an environmental sensor system configured to determine locations for placement of pollutant sensors, according to aspects of the present disclosure.

FIG. 2 shows a diagram of an example of a geographic region for placement of pollutant sensors, according to aspects of the present disclosure.

FIG. 3 shows a diagram of an example placement of pollutant sensors using the system of FIG. 1, according to aspects of the present disclosure.

FIG. 4 shows a flowchart of an example method of determining locations for pollutant sensors, according to aspects of the present disclosure.

FIG. 5 is a block diagram of another example of an environmental sensor system configured to determine locations for placement of pollutant sensors, according to aspects of the present disclosure.

FIG. 6 is a diagram of an example elevation profile of a geographic region for placement of pollutant sensors, according to aspects of the present disclosure.

FIG. 7 is a diagram of an example facility map within a geographic region for placement of pollutant sensors, according to aspects of the present disclosure.

FIG. 8 is a diagram of an estimated emissions probability map for a geographic region for placement of pollutant sensors, according to aspects of the present disclosure.

FIG. 9 shows a flowchart of another example method of determining locations for pollutant sensors, according to aspects of the present disclosure.

FIG. 10 is a block diagram illustrating example physical components of a computing device with which aspects of the disclosure may be practiced.

FIGS. 11 and 12 are simplified block diagrams of a mobile computing device with which aspects of the present disclosure may be practiced.

### DETAILED DESCRIPTION

In the following detailed description, references are made to the accompanying drawings that form a part hereof, and in which are shown by way of illustrations specific aspects or examples. These aspects may be combined, other aspects may be utilized, and structural changes may be made without departing from the present disclosure. Aspects may be practiced as methods, systems, or devices. Accordingly, aspects may take the form of a hardware implementation, an entirely software implementation, or an implementation combining software and hardware aspects. The following detailed description is therefore not to be taken in a limiting sense, and the scope of the present disclosure is defined by the appended claims and their equivalents.

The present disclosure describes various examples of a computing device that determines locations for placement of pollutant sensors. For example, the computing device receives data from pollutant source, environmental sensors, and/or external data sources and determines suitable locations for placement of pollutant sensors. In some aspects, the computing device also determines a number of pollutant sensors to be placed and/or characteristics of the pollutant sensors. The characteristics may include detection type, pollutant detection sensitivities, spatial resolution, temporal resolution, purchase cost, maintenance cost, and/or other suitable characteristics. In some scenarios, the pollutant sensors detect the presence or measure levels of pollutants such as methane or other greenhouse gases.

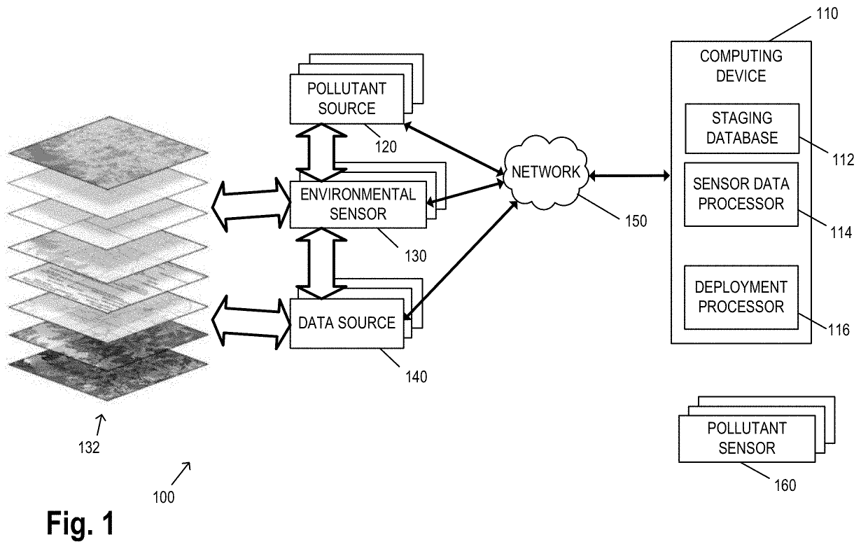

This and many further aspects of a computing device are described herein. For instance, FIG. 1 shows a block diagram of an example of an environmental sensor system 100 (hereinafter, “system 100”) configured to determine locations for placement of pollutant sensors 160, according to an example embodiment. The environmental sensor system 100 comprises a computing device 110, one or more pollutant sources 120, and one or more environmental sensors 130. In some aspects, the system 100 also includes one or more data sources 140. At least some of the computing device 110, pollutant sources 120, environmental sensors 130, and data sources 140 may be communicatively coupled by a network 150.

Computing device 110 may be any type of computing device, including a mobile computer or mobile computing device (e.g., a Microsoft® Surface® device, a laptop computer, a notebook computer, a tablet computer such as an Apple iPad™, a netbook, etc.), or a stationary computing device such as a desktop computer or PC (personal computer). In some aspects, computing device 110 is a network server, cloud server, or other suitable distributed computing system. Computing device 110 may be configured to execute one or more software applications (or “applications”) and/or services and/or manage hardware resources (e.g., processors, memory, etc.), which may be utilized by users (e.g., customers) of the computing device 110.

The pollutant sources 120 may comprise ground locations, buildings, equipment (e.g., fixed industrial equipment), that may leak one or more pollutants, in various aspects. In general, a pollutant as used herein is a substance that has undesired effects on an environment, or adversely affects the usefulness of a resource or device. In some aspects, the pollutants may include greenhouse gases (GHG) such as methane, nitrous oxide, ozone, carbon dioxide, or other gases that contribute to a “greenhouse effect.” In other aspects, the pollutants may include carbon monoxide, hydrocarbons, other gases, liquids (e.g., gasoline, kerosene), vapors, aerosols, particulate matter (e.g., soot from internal combustion engines, coal dust), or other chemicals and compounds. In still other aspects, the pollutants may be other substances, even those generally considered beneficial (such as breathable oxygen or potable water), that are being emitted in an undesirable area, such as water leaks from a device that generates or filters water, or oxygen leaks from a pressurized habitat for humans. In some embodiments, the pollutant sources 120 are carbon capture, utilization, and storage (CCUS) sites and the pollutant sensors 160 are placed for leak monitoring and/or air quality monitoring for volatile organic compounds or other pollutants.

For ease of discussion, the aspects herein are described with methane as a pollutant. Accordingly, the pollutant sources 120 may include oil and gas industry infrastructure (e.g., equipment and industrial sites for production, processing, transmission, storage, and distribution), as well as agricultural sites, landfills, and abandoned oil and gas fields. Examples of oil and gas infrastructure include pipelines, tank batteries, compressor stations, well pads, their supporting equipment, and the like.

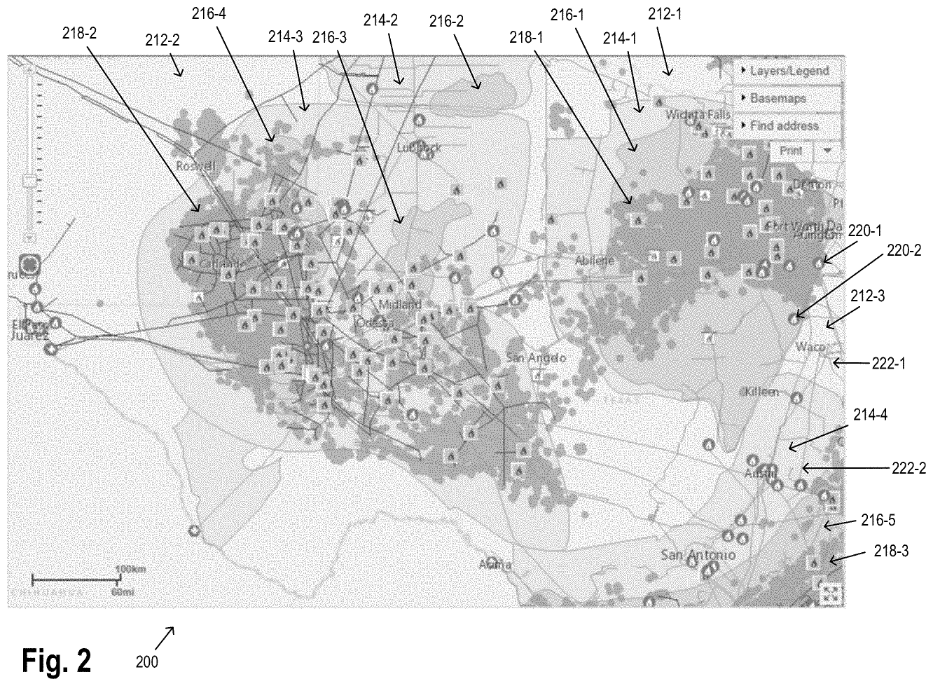

The pollutant sources 120 are generally spread out over a geographic region or region of interest, such as geographic region 200 shown in FIG. 2. Each pollutant source 120 may leak pollutants at varying levels and times. Generally, emissions from different pollutant sources 120 may have different characteristics when analyzed using time series data. The emissions from pneumatic controllers, chemical injection pumps, and/or from water flashing (e.g., release of methane dissolved in water when released to the atmosphere) may exhibit short term temporal variability, long term variability, and/or diurnal periodicity, in various scenarios. For example, a pollutant source 120 may emit a constant, low level of methane over time. As another example, where the pollutant source 120 is a compressor that compresses methane into liquid form, it may have an intermittent, moderate level leak that begins when pressure exceeds a limit of a valve seal. As yet another example, where the pollutant source 120 is a pipeline that transfers methane gas, it may have a leak that starts after a long period of time without leaks (e.g., beginning to leak after several years in service). Still other pollutant sources 120 may be designed to never leak or exceed design parameters for leakage, but may still be monitored for leaks and/or excessive leakage due to deterioration, accidents (e.g., collisions with other equipment), vandalism, etc.

The environmental sensors 130 are sensors that measure and/or detect the presence of an environmental characteristic and provide corresponding data, such as various layers of environmental map data 132. Generally, the environmental sensors 130 provide data related to environmental characteristics within the geographic region 200, or nearby regions that may affect dispersion of a pollutant within the geographic region 200. In some aspects, environmental sensors 130 may be carried to a sensing location by a vehicle, such as a satellite, drone, plane, helicopter, atmospheric research vehicle, or other suitable vehicle. In other aspects, the environmental sensors 130 are provided in fixed locations.

In various aspects, the environmental sensors 130 measure and/or detect environmental characteristics related to dispersion of the pollutants emitted by the pollutant sources 120. Where the pollutant is methane, for example, the environmental sensors 130 may measure and/or detect one or more of reflected sunlight or laser light (in ultraviolet, visible, infrared, and/or short-wave infrared), ambient temperature, humidity, wind speed and direction, and other suitable environmental characteristics. In some aspects, the environmental sensors 130 provide different measurements at two or more heights (e.g., with multiple readings at different times), for example, at ground level, cloud level, every 50 meters, etc. Moreover, some environmental sensors 130 may provide data with different temporal resolutions, for example, providing measurements in real-time, five minute intervals, every four hours, daily, monthly, etc. In the aspects described herein with respect to methane as the pollutant, the environmental sensors 130 may comprise optical gas imaging cameras (forward looking infrared, nondispersive infrared), catalytic detection sensors, satellites, aerial surveys, Internet of Things (IoT) sensor grids, unmanned vehicles (e.g., aerial drones or ground-based vehicles), fixed sensors attached to the pollutant sources 120, or other suitable sensors.

Some environmental sensors 130 may be located within a vicinity of, coupled with, or integral to a pollutant source 120. For example, an environmental sensor 130 that measures ambient temperature and pressure may be integral to a compressor (i.e., pollutant source 120) that compresses methane into liquid form. As another example, an environmental sensor that measures methane levels may be located within 5 meters, 20 meters, or another suitable distance from a well pad. Some environmental sensors 130 may be standalone or independent sensors, such as hand-held optical gas imaging cameras or soil moisture sensors. Some environmental sensors 130 may be part of a sensor package that combines several sensors, for example, a sensor package that measures temperature, wind speed, and wind direction. In some aspects, the environmental sensors 130 are small, inexpensive sensors, such as Internet of Things sensors, that form a mesh of data streams that may be analyzed in near real-time to accurately detect anomalous emissions and identify the source of leaks.

Aerial imagery or data may be captured using cameras, light detection and ranging (LiDAR) equipment, gas spectrometers, or other suitable detectors as the environmental sensors 130. This data may be available monthly, or at 3-month intervals, other suitable intervals, or on demand, for example, due to generally higher operating costs (e.g., for fuel, pilot fees, aircraft maintenance, etc.). Unmanned vehicles may utilize laser absorption spectroscopy, cavity-enhanced laser spectroscopy, or other suitable sensors as the environmental sensors 130. In an embodiment, an autonomous or semi-autonomous drone may fly along a pre-planned path and gather data near the ground at a regular cadence, for example, along a length of a pipeline.

The data sources 140 comprise databases, data sets, and/or compilations of data representing environmental characteristics, including detected pollutants, measured levels of pollutants, reported pollutant leaks, etc. In some aspects, the data sources 140 receive data from at least some of the environmental sensors 130 (e.g., satellites, aerial surveys, sensors at pollutant sources 120). The data sources 140 may also utilize data from mapping services that provide geographical maps, topographical maps, ground cover maps, etc. Access to the data sources 140 may be provided by a government, commercial business or service, non-profit group, or other entity. In some aspects, the data sources 140 include a mapping service that provides geographical maps, topographical maps, ground cover maps, etc.

In some embodiments, the data sources 140 may comprise one or more of a land-use data source, pollutant facility data source, or a data source provided by the European Space Agency (e.g., providing Sentinel 2 data, Sentinel-5P data), National Aeronautics and Space Administration (Terra MODIS and Aqua MODIS data), GHGSat, a weather service (e.g., National Weather Service), National Oceanic and Atmospheric Administration (NOAA Operational Model Archive and Distribution System), Emissions Database for Global Atmospheric Research (EDGAR), United States Geological Survey (e.g., Landsat 8 data), Total Carbon Column Observing Network (TCCON), oil and gas production companies (e.g., ExxonMobil, Chevron), New Mexico Oil Conservation Division (e.g., providing reports of methane emissions), Texas Rail Road Commission (e.g., providing reports of methane emissions), and/or other suitable data source providers.

The computing device 110 includes a staging database 112, a sensor data processor 114, and a deployment processor 116. Generally, the computing device 110 is configured to receive data from the pollutant source 120, the environmental sensors 130, and/or the data sources 140 and determine suitable locations for placement of pollutant sensors 160. More specifically, the staging database 112 receives data from the pollutant sources 120, the environmental sensors 130, and/or the data sources 140. Data from the pollutant source 120 may include diagnostic and control information. For example, operators of the pollutant sources 120 (e.g., an energy production company, chemical production company, refinery) and/or control systems of the pollutant sources 120 may provide Supervisory Control And Data Acquisition (SCADA) data or other suitable information to the staging database 112. In some scenarios, the computing device 110 is configured to actively request updates from some of the pollutant sources 120, the environmental sensors 130, and/or the data sources 140, for example, on a suitable schedule (e.g., every week, every month). In other scenarios, the computing device 110 requests the updates in response to a user request.

The sensor data processor 114 is configured to perform processes that improve compatibility among data from different data sources 140 and different environmental sensors 130. For some of the data sources 140, the sensor data processor 114 may be configured to process data within the staging database 112 (i.e., after the data is received by the staging database 112) to have a common format, organization, etc. For some of the data sources 140, the sensor data processor 114 may be configured to receive and process data from the data sources 140 before storing the data within the staging database 112.

In some aspects, the sensor data processor 114 processes and/or transforms data into common data having a common format, common “grid lines” within the geographic region 200 (e.g., regular grids, rectilinear grids, curvilinear grids), common temporal resolution (e.g., data is present at 1 hour intervals), common spatial resolution (e.g., data is present for each 50 m² area), and/or common elevation resolution (e.g., data is present for each 50 m elevation interval). In some scenarios, the sensor data processor 114 scales data to a more coarse format (e.g., scaling 5 minute data to 20 minute data). In other scenarios, the sensor data processor 114 may interpolate existing data to generate new data to improve the compatibility. For example, a first set of data may have a temporal resolution of 5 minutes, while a second set of data may have a temporal resolution of 20 minutes, and the sensor data processor 114 may interpolate the second set of data to generate equivalent data at the temporal resolution of 5 minutes. As another example, a third data set may have a spatial resolution of 50 meters, while a fourth data set may have a spatial resolution of 250 meters, and the sensor data processor 114 may interpolate the fourth set of data to generate equivalent data at the spatial resolution of 50 meters. The sensor data processor 114 may be configured to perform bilinear interpolation, nearest neighbor interpolation, inverse distance interpolation, and/or other suitable interpolation techniques, in various aspects.

The deployment processor 116 is configured to determine locations within a geographic region (e.g., geographic region 200) for placement of the pollutant sensors 160 based on environmental characteristics (e.g., the data within the staging database 112). In some embodiments, the deployment processor 116 determines one or more of a number of pollutant sensors 160, locations of the pollutant sensors 160, and/or characteristics of the pollutant sensors 160. As one example, the deployment processor 116 determines a number of pollutant sensors 160 and locations for their placement to meet a desired level of detectability (e.g., detect leaks greater than 5 kg per hour) at a desired cost (e.g., $100,000 for sensors and installation). As another example, the deployment processor 116 determines a first number of high sensitivity sensors, a second number of low sensitivity sensors, and their respective locations to meet a desired level of detectability at a desired cost. Example characteristics of the pollutant sensors 160 may include detection type (e.g., infrared, gas spectrometry, etc.), pollutant detection sensitivities (e.g., 100 parts per million, 500 parts per million, etc.), spatial resolution (50 meters, 1000 meters, etc.), temporal resolution (real-time, 5 minute intervals, 4 hours, daily, monthly), purchase cost, maintenance cost, and/or other suitable characteristics.

Generally, the deployment processor 116 is configured to determine a sensor configuration (e.g., the number, locations, and/or characteristics of the pollutant sensors 160) to detect pollutant leaks or related anomalies. In some scenarios, the deployment processor 116 determines an optimal sensor configuration for a given set of constraints (e.g., price, detectability of the pollutant, etc.). The deployment processor 116 may be configurable to determine a sensor configuration based on adjustable criteria, for example, a number of simultaneous leaks that may be detected, a severity of leaks that can be detected (e.g., 1 kg/hr, 100 kg/hr), time until a leak is detected, likelihood of detection for leaks that exceed a safety threshold (e.g., likelihood of detecting a 100 kg/hr leak) within a predetermined time period (e.g., within 2 hours, 3 days, etc.), cost of the pollutant sensors 160, installation cost of the pollutant sensors 160, whether adjacent equipment (i.e., pollutant sources 120) need to be turned off or disabled for installation and/or maintenance of the pollutant sensor 160, and/or other suitable criteria. For example, a moderate leak may take several hours to reach detectable levels of a low-cost 1000 part per million (ppm) pollutant sensor 160, while a more expensive 100 ppm pollutant sensor 160 may detect the moderate leak within 10 minutes. Moreover, a large network of inexpensive pollutant sensors 160 may require a large labor cost for installation and maintenance, while similar performance in detectability may be achieved with fewer, but higher quality pollutant sensors 160. The deployment processor 116 may also select a pollutant sensor 160 based on its suitability for a particular sub-region in which it will be placed (i.e., an operating environment). For example, optical gas imaging cameras may be less susceptible to sensor poisoning but more sensitive to moisture and are generally more expensive to operate than a catalytic detection sensor, which may be subject to sensor poisoning but be less expensive to purchase and can resist higher humidity.

The deployment processor 116 may be configured to determine a sensor configuration where a set of leaks to be detected is as disjoint as possible (i.e., spread out over a large area within geographic region 200), but where the set of leaks to be detected also contains the biggest leaks that may occur. In some scenarios, the deployment processor 116 determines a new sensor configuration based on an existing sensor configuration and adds new pollutant sensors 160, moves existing pollutant sensors 160, and/or replaces existing pollutant sensors 160 with different models. In some scenarios, there may be a preferred minimum distance between a sensor and equipment the sensor is configured to monitor, for example, to allow for installation, maintenance, and/or replacement of the pollutant sensor 160 without disrupting operation of the equipment itself (e.g., turning off the equipment to install the pollutant sensor 160).

In some aspects, the deployment processor 116 determines flight paths for drones and/or aerial vehicles that carry the pollutant sensors 160, orbital paths for satellites that carry the pollutant sensors 160, and/or ground paths for wheeled or tracked vehicles that carry the pollutant sensors 160. The flight paths, orbital paths, and/or ground paths may be determined based on a desired frequency of data (i.e., daily or weekly flyovers), cost of operation, or other suitable criteria.

Although only one instance of the computing device 110 is shown, several instances of the computing device 110 may be utilized, in various aspects. For example, the computing device 110 may be part of a distributed computing system and cooperate with other instances of the computing device 110 (not shown) to perform the steps described herein. In other aspects, the staging database 112, the sensor data processor 114, and/or the deployment processor 116 may be distributed across one, two, three, or more instances of the computing device 110. For example, a first instance of the computing device 110 may include the staging database 112, a second instance of the computing device 110 may include the sensor data processor 114, and third and fourth instances of the computing device 110 may include instances of the deployment processor 116.

The pollutant sensors 160 are configured to detect the presence (e.g., detected, not detected), rate of emission (e.g., kg per hour), and/or concentration level (e.g., number of particles in parts per million in a given volume or mass of particles) of pollutants. The pollutant sensors 160 may be selected from a plurality of available sensors having different pollutant detection sensitivities, spatial resolutions, temporal resolutions, purchase price, installation price, maintenance price, etc.

Network 150 may comprise one or more networks such as local area networks (LANs), wide area networks (WANs), enterprise networks, the Internet, etc., and may include one or more of wired and/or wireless portions. Computing device 110, pollutant source 120, environmental sensors 130, and data sources 140 may include at least one wired or wireless network interface that enables communication with each other (or an intermediate device, such as a Web server or database server) via network 150. Examples of such a network interface include but are not limited to an IEEE 802.11 wireless LAN (WLAN) wireless interface, a Worldwide Interoperability for Microwave Access (Wi-MAX) interface, an Ethernet interface, a Universal Serial Bus (USB) interface, a cellular network interface, a Bluetooth™ interface, or a near field communication (NFC) interface.

FIG. 2 shows a diagram of an example of a geographic region 200 for placement of pollutant sensors 160, according to an example embodiment. In this example, the geographic region 200 represents a portion of the Permian basin in Texas and New Mexico. In various aspects, the deployment processor 116 is configured to divide a geographic region into a plurality of sub-regions based on environmental characteristics within the geographic region. For example, the deployment processor 116 may divide the geographic region 200 into sub-regions and/or groups of sub-regions, such as sub-region group 212 (including sub-regions 212-1, 212-2, 212-3), sub-region group 214, sub-region group 216, and sub-region group 218 based on the data within the staging database 112. The deployment processor 116 divides the geographic region 200 into areas with similar pollutant-related environmental characteristics, such as wind speed, wind direction, temperature, humidity, cloud cover, land use (e.g., buildings and structures such as well pads 220 and pipelines 222), elevation, and other suitable characteristics.

In some aspects, the deployment processor 116 identifies a sub-region as a contiguous cluster of areas that are within the geographic region 200 and have common (e.g., similar) dispersion effects on the pollutant. For example, a sub-region that encompasses a valley may generally channel methane down the valley in a direction consistent with typical wind flows. In some aspects, the identified sub-region has the common dispersion effects on the pollutant over different seasons. In other words, the deployment processor 116 is configured to identify sub-regions having similar dispersion effects for an entire season (i.e., spring, summer, fall, winter, dry season, monsoon season, etc.), or several seasons. In another embodiment, the deployment processor 116 identifies sub-regions having similar characteristics over a fixed period, such as 1 month, 3 months, 6 months, 12 months, etc. These approaches provide for improved sensor placement in that a location of a pollutant sensor 160 will be more relevant for a longer duration of the year, as opposed to extreme weather events, for example, only during or shortly after a rain storm or high wind event. In other words, the deployment processor 116 identifies time-varying trends in the environmental characteristics and identifies the sub-regions based on the time-varying trends for improved consistency in sensor placement.

The deployment processor 116 may include a neural network model (not shown) that processes the data within the staging database to divide the geographic region 200 into sub-regions and determine locations for placement of the pollutant sensors 160. In some aspects, the neural network model is a convolutional neural network model, an autoencoder, time-modeling neural network (e.g., NeuralProphet), or other suitable neural network model. The neural network model may be configured to receive data from the staging database 112 and identify likely locations for pollutant leaks based on historical leak data and environmental characteristic data. For example, map data associated with a detected pollutant leak in a geographic region may be correlated with atmospheric data and/or topographical data to identify trends and/or patterns in dispersion of the pollutant.

In some embodiments, the neural network model is a deep neural network for spatial regression or classification. Examples of such networks include fully convolutional networks (e.g., U-net networks, memory-less convolutional networks), transformer-based networks, recurrent neural networks (e.g., long short-term memory networks), or other suitable networks.

In some aspects, the deployment processor 116 utilizes the neural network model to generate a stream of pollutant dispersion predictions (e.g., an estimated dispersion of the pollutant through the plurality of sub-regions), such as maps showing locations and concentrations of methane over a suitable time period (e.g., every 6 hours over 72 hours, every hour over 36 hours, etc.). The stream of pollutant dispersion predictions may be validated against measured and/or reported leak data within the staging database 112 for training the neural network model, for example.

The deployment processor 116 may utilize a centroid model, for example, a k-means algorithm that represents each cluster by a single mean vector, to identify sub-regions having similar environmental characteristics and/or pollutant dispersion effects. Each sub-region may belong to one or more groups of sub-regions where each of groups of sub-regions have common dispersion effects on the pollutant. At least some sub-regions within a same group may be non-contiguous. For example, sub-regions 212-1, 212-2, and 212-3 represent sub-regions within a same sub-region group 212 and are not contiguous with each other.

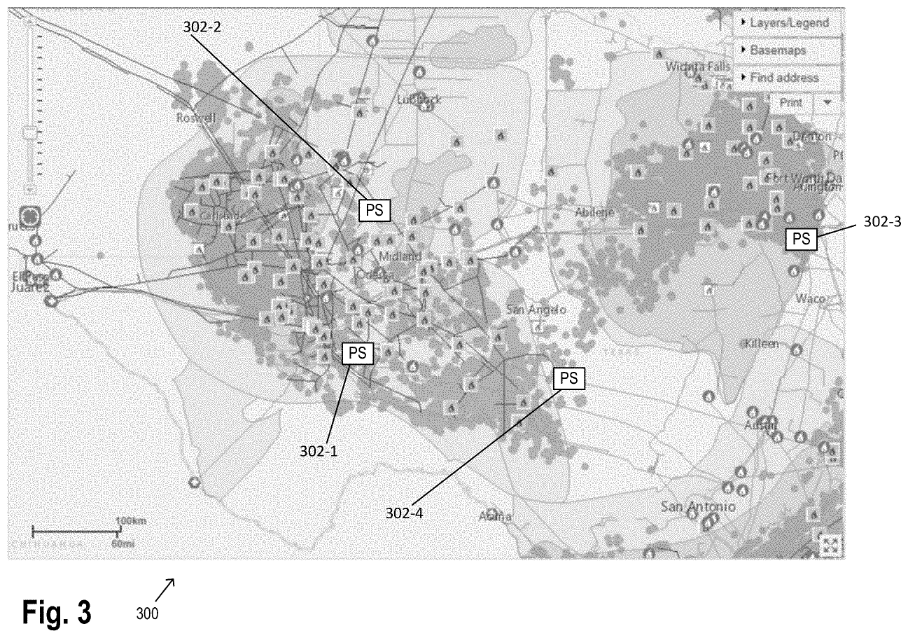

FIG. 3 shows a diagram of an example placement of pollutant sensors 302 using the system 100 of FIG. 1, according to an example embodiment. In the embodiment shown in FIG. 3, the pollutant sensors 302 are generally placed downwind from closely spaced groups of pollutant sources, such as well pads 220. In some aspects, the deployment processor 116 performs a validation of the locations for sensor placement by performing a simulation using probabilities for pollutant emissions based on historical emissions. For example, the deployment processor 116 may utilize GHGSat data for detected methane leaks and state databases for reported methane leaks to determine whether or not the methane leaks are detected, how quickly the methane leaks are detected, etc.

In some aspects, the deployment processor 116 is configured to place a number n of “virtual” instances of the pollutant sensors 160 into the geographic region 200 and randomly (or pseudo-randomly) select a subset of x pollutant sensors 160 to be used to predict measured values from the remaining (n−x) pollutant sensors 160. After simulating different configurations of sensors for the subset of x pollutant sensors 160, the deployment processor 116 selects the configuration that has a lowest prediction error. This implies that the selected subset of x pollutant sensors 160 in the minimal error configuration, is generally representative of the geographic region while using fewer sensors, providing an improved “value for data” perspective.

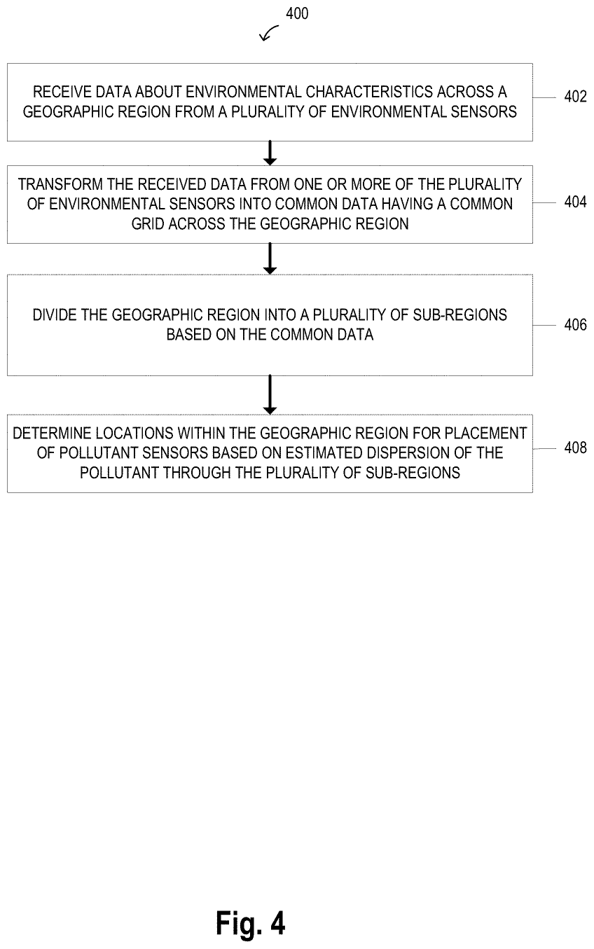

FIG. 4 shows a flowchart of an example method 400 of determining locations for pollutant sensors, according to an example embodiment. Technical processes shown in these figures will be performed automatically unless otherwise indicated. In any given embodiment, some steps of a process may be repeated, perhaps with different parameters or data to operate on. Steps in an embodiment may also be performed in a different order than the top-to-bottom order that is laid out in FIG. 4. Steps may be performed serially, in a partially overlapping manner, or fully in parallel. Thus, the order in which steps of method 400 are performed may vary from one performance to the process of another performance of the process. Steps may also be omitted, combined, renamed, regrouped, be performed on one or more machines, or otherwise depart from the illustrated flow, provided that the process performed is operable and conforms to at least one claim. The steps of FIG. 4 may be performed by the computing device 110 (e.g., via the sensor data processor 114 and/or the deployment processor 116), or other suitable computing device.

Method 400 begins with step 402. At step 402, data about environmental characteristics across a geographic region is received from a plurality of environmental sensors. The geographic region includes one or more pollutant sources that emit a pollutant. The geographic region corresponds to the geographic region 200 and the data may be received, at the staging database 112 and/or the sensor data processor 114, from the pollutant sources 120, environmental sensors 130, and/or data sources 140, in various aspects.

At step 404, the received data from one or more of the plurality of environmental sensors is transformed into common data having a common grid across the geographic region. In an embodiment, for example, the sensor data processor 114 transforms the data within the staging database 112, as described above, to regrid the data to a common grid (e.g., a same regular grid, rectilinear grid, or curvilinear grid). In some aspects, the sensor data processor 114 transforms (e.g., regrids) the received data by interpolating first data about the environmental characteristics from a first data source to generate second data that is aligned with the common grid, where the common grid is associated with a second data source. For example, the sensor data processor 114 may transform first data having a rectilinear grid to have a curvilinear grid that matches second data. Interpolating may include one or both of a temporal interpolation and a spatial interpolation. The first data source and the second data source may be selected from a land-use data source, a meteorological data source, a pollutant facility data source, a satellite-based pollutant emissions data source, or other suitable data sources, such as the data sources 140.

At step 406, the geographic region is divided into a plurality of sub-regions based on the common data. In an embodiment, for example, the deployment processor 116 divides the geographic region 200 into the sub-region groups 212, 214, 216, and 218 based on the environmental characteristics within the staging database 112. In some aspects, dividing the geographic region comprises identifying a sub-region as a contiguous cluster of areas that are within the geographic region and have common dispersion effects on the pollutant. Each of the plurality of sub-regions may belong to a sub-region group of a plurality of sub-region groups, each sub-region within a sub-region group having common dispersion effects on the pollutant. In some aspects, the common dispersion effects on the pollutant occur over different seasons. In some scenarios, at least some sub-regions within a same sub-region group are non-contiguous. For example, first and second sub-regions within a same group may be separated from each other by a third sub-region within a different group. Moreover, a sub-region may be completely surrounded or encapsulated by another sub-region (e.g., a sub-region corresponding to a hill-top or other raised elevation feature.

At step 408, locations within the geographic region are determined for placement of pollutant sensors based on estimated dispersion of the pollutant through the plurality of sub-regions. In an embodiment, the deployment processor 116 determines locations of the pollutant sensors 160 within the geographic region 200, as described above. In some aspects, the deployment processor 116 determines a number of the pollutant sensors 160 for the placement and the locations of the pollutant sensors 160 that provide a minimum pollutant detectability threshold. The pollutant sensors 160 may be selected from a plurality of pollutant sensors having different pollutant detection sensitivities.

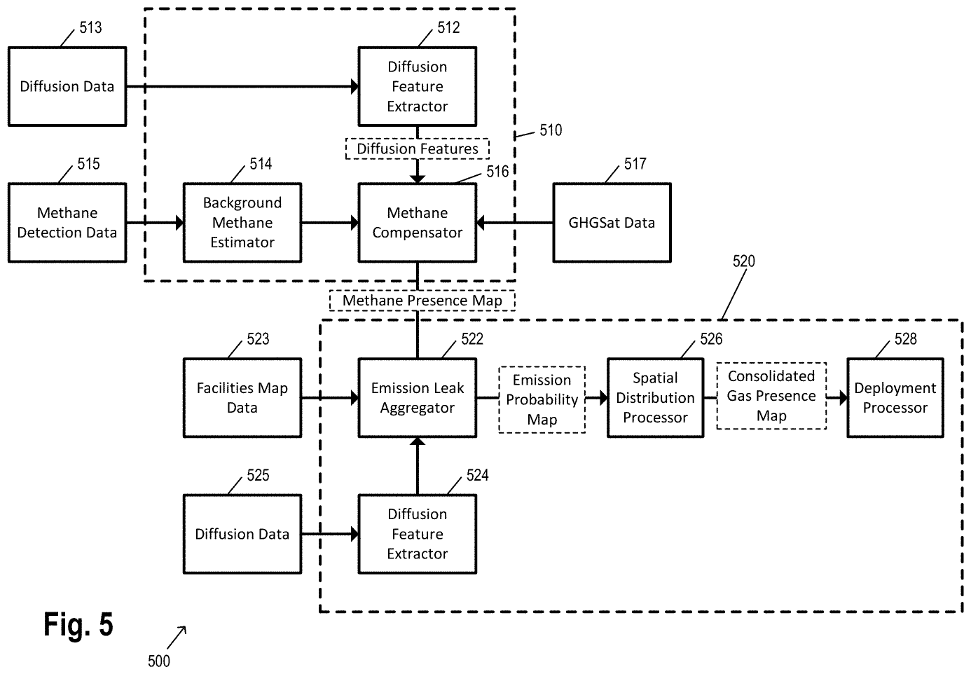

FIG. 5 is a block diagram of another example of an environmental sensor system 500 (hereinafter, “system 500”) configured to determine locations for placement of pollutant sensors 160, according to aspects of the present disclosure. The system 500 comprises a pollutant reference module 510 and a sensor placement module 520. The pollutant reference module 510 is configured to determine a reference concentration level of a pollutant within a geographic region, such as methane within the geographic region 200. The reference concentration level may then be compared to measured levels to facilitate the identification of pollutant leaks. The sensor placement module 520 determines locations for placement of the pollutant sensors 160, as described herein. In some embodiments, one or more elements of the system 500 are included within the computing device 110. For example, the pollutant reference module 510 may be included within the sensor data processor 114 and/or the sensor placement module 520 may be included within the deployment processor 116.

The pollutant reference module 510 is configured to combine a remote sensing (e.g., satellite or aerial based) spatial measurement of excess methane presence over an area of interest (e.g., from GHGSat as the data source 140) with estimates of naturally occurring methane from published datasets (e.g. from Sentinel-5P TROPOMI as the data source 140) and possible methane leaks from neighboring oil and gas infrastructure (e.g., as pollutant source 120). A methane presence map of the area of interest with human-generated methane presence (as opposed to naturally occurring methane) is generated based on the combination. This methane presence map provides a reference when configuring and operating an emission leak aggregator, such as emission leak aggregator 522, described below. In various embodiments, the pollutant reference module 510 generates a methane presence map for monthly time intervals, for example, sequential maps for January, February, March, etc. In other embodiments, the pollutant reference module 510 generates methane presence maps for a selected time window, for example, sequential maps every hour over a time window of four weeks, etc.

The pollutant reference module 510 comprises a diffusion feature extractor 512, a background methane estimator 514, and a methane compensator 516. The diffusion feature extractor 512 is configured to model methane entering the geographic area 200 from oil and gas infrastructure that are nearby, but outside of the geographic area 200. The diffusion feature extractor 512 receives diffusion data 513 from environmental sensors 130 and/or data sources 140, such as digital elevation models (DEMs), climatology variables such as wind speed, direction, etc., and identifies diffusion features affecting dispersion of methane from the oil and gas infrastructure outside of the geographic area 200.

The background methane estimator 514 receives background methane estimates (methane detection data 515) from data sources 140 for the geographic region, selects components of the background methane estimates that are due to natural factors (e.g., excluding those from leaking infrastructure), and adjusts a spatial scale of the background methane estimates based on the size of the geographic region. The methane detection data 515 may include Sentinel-5P TROPOMI data, EDGAR data, or other suitable background methane data.

The methane compensator 516 is configured to transform data for natural background methane (from background methane estimator 514) and influx of methane due to neighboring pollutant sources 120 (from GHGSat data 517) using diffusion features of excess methane (estimated by the diffusion feature extractor 512) to generate the reference concentration level of the pollutant within the geographic region 200.

The sensor placement module 520 comprises an emission leak aggregator 522, a diffusion feature extractor 524, a spatial distribution processor 526, and a deployment processor 528. The diffusion feature extractor 524 is configured to model methane entering the geographic area 200 from oil and gas infrastructure that are within the geographic area 200. The diffusion feature extractor 524 receives diffusion data 525 from environmental sensors 130 and/or data sources 140, such as digital elevation models (DEMs), climatology variables such as wind speed, direction, etc., and identifies diffusion features affecting dispersion of methane from the oil and gas infrastructure within the geographic area 200.

The emission leak aggregator 522 may be a deep learning neural network model that is trained based on oil & gas facilities coordinates (e.g., facilities map data 523 including coordinate data for locations of pollutant sources 120) and type information (e.g., well pads, pipelines, etc.). In some scenarios, the emission leak aggregator 522 is also trained with satellite data or other suitable data from environmental sensors 130, for example, to estimate a probability of a facility leaking a given pollutant. The training of the emission leak aggregator 522 may use remote sensing devices (e.g., satellite or aerial based environmental sensors) that cover large areas and train the emission leak aggregator 522 to generate an emission probability map for the pollutant based on the reference concentration level of the pollutant (from the methane compensator 516), the facilities map data 523, and the diffusion features (from the diffusion feature extractor 524).

The spatial distribution processor 526 is configured to divide the geographic region 200 into a plurality of sub-regions according to common dispersion effects, as described above. In some embodiments, the spatial distribution processor 526 is a deep learning neural network model trained on process model simulations, such as the Weather Research and Forecasting (WRF) model or CALPUFF model or other atmospheric models which model a methane plume (or puff) as it disperses over time. The plume dispersion (or puff dispersion) over time (e.g., over a 24 hour period, 48 hour period, etc.) may identify locations where the pollutant sensors 160 should be placed to detect the most leaks, the worst leaks, etc. In some scenarios, the plume dispersion may indicate suitable locations for a pollutant sensor 160, such as localized areas of relatively high concentration of the pollutant due to a leak, localized areas of relatively long duration of dispersion of the pollutant, and/or localized areas that receive methane from multiple different pollutant sources 120.

The spatial distribution processor 526 may use a heuristic-based clustering approach, for example, using a clustering algorithm, such as K-Means or Gaussian Mixture Model or DBScan, to identify clusters in the geographic region 200 having common dispersion effects, based on diffusion datasets (e.g., diffusion data 513, 525), oil and gas infrastructure datasets (e.g., facilities map data 523), and methane leak data (e.g., GHGSat data 517). By grouping regions with common dispersion effects, a smaller number of sensors may be placed within the geographic region 200 to provide a suitable level of pollutant detectability, which enables optimization of costs as well as detectability of a highest number of leaks and/or worst leaks. The identified clusters are included in a consolidated gas presence map generated by the spatial distribution processor 526. In some embodiments, the clusters correspond to the sub-region group 212, sub-region group 214, sub-region group 216, and sub-region group 218 as shown in FIG. 2.

The deployment processor 528 is configured to determine locations for placement of the pollutant sensors 160 by combining sensor sensitivity information of the pollutant sensors 160, a budget for a number of sensors that can be placed in the geographic area, and the gas presence map from the spatial distribution processor 526. In some scenarios, the locations may be provided as a sensor placement map, generated by the deployment processor 528, such that a likelihood of detecting a leak is maximized for the given budget. In other scenarios, the deployment processor 528 provides an array of coordinates within the geographic region that correspond to locations where a pollutant sensor 160 should be placed.

The deployment processor 528 combines the clusters (e.g., from the spatial distribution processor 526) and the facilities map data 523 and identifies locations within each cluster that maximizes a probability of detecting a leak from pollutant sources 120 within each cluster for a given number of sensors. The deployment processor 528 may use a heuristic-based solution or an optimization-solution to iterate over placing the pollutant sensors 160 in different locations within each cluster, for example. In some scenarios, the deployment processor 528 is configured for different sensor placement goals, such as being able to identify which oil and gas infrastructure component caused a particular leak. In one such scenario, the inputs are the same but the goal of the sensor placement strategy and hence the underlying optimization or heuristic routine are adapted to this new problem.

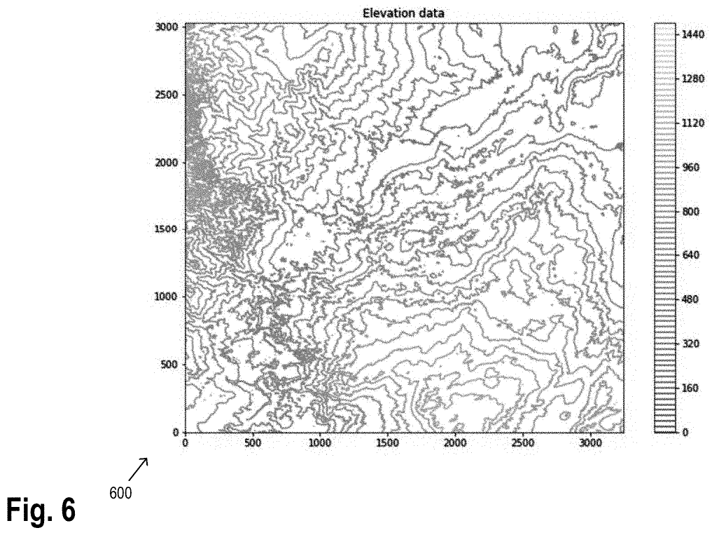

FIG. 6 is a diagram of an example elevation profile 600 of a geographic region for placement of pollutant sensors, according to aspects of the present disclosure. The elevation profile 600 is an example of a digital elevation models, such as digital elevation models within the diffusion data 513 and 525.

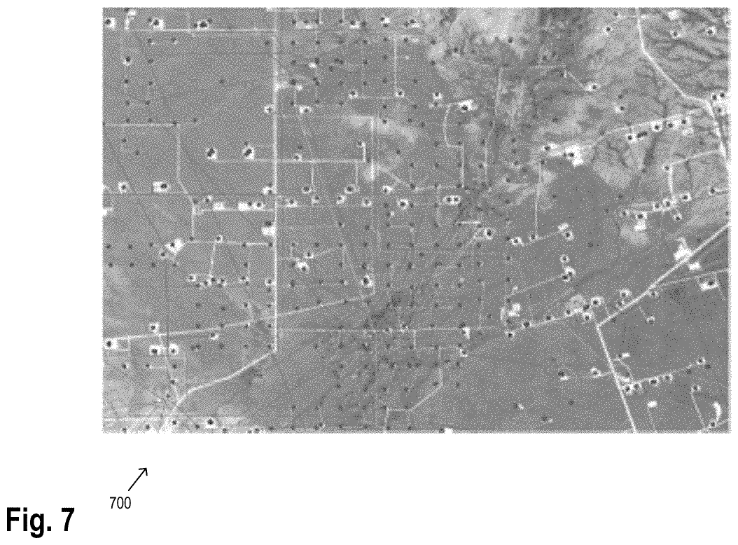

FIG. 7 is a diagram of an example facility map 700 within a geographic region for placement of pollutant sensors, according to aspects of the present disclosure. The facility map 700 is a satellite image based map that is overlaid with oil and gas infrastructure (well locations and pipelines) in a portion of the geographic region 200.

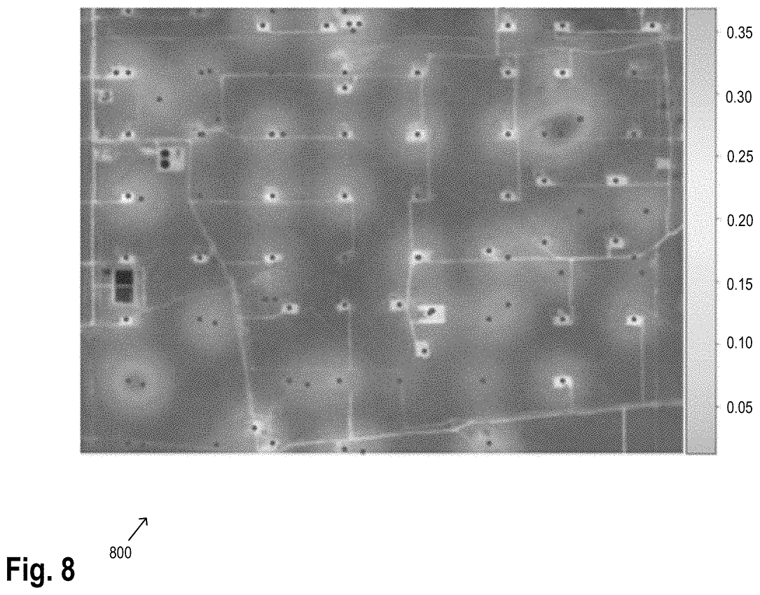

FIG. 8 is a diagram of an estimated emissions probability map 800 for a geographic region for placement of pollutant sensors 160, according to aspects of the present disclosure. The estimated emissions probability map 800 is a heat map of emissions probability and, in the example shown in FIG. 8, is overlaid on a satellite image map showing oil and gas facilities (e.g., oil and gas wells). In some embodiments, the estimated emissions probability map 800 is generated by the emission leak aggregator 522.

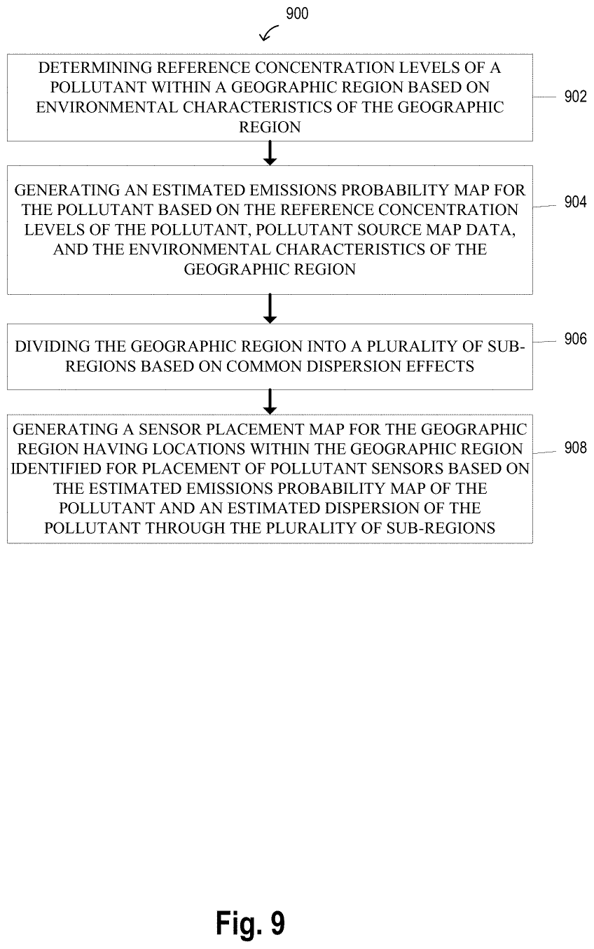

FIG. 9 shows a flowchart of another example method 900 of determining locations for pollutant sensors, according to an example embodiment. Technical processes shown in these figures will be performed automatically unless otherwise indicated. In any given embodiment, some steps of a process may be repeated, perhaps with different parameters or data to operate on. Steps in an embodiment may also be performed in a different order than the top-to-bottom order that is laid out in FIG. 9. Steps may be performed serially, in a partially overlapping manner, or fully in parallel. Thus, the order in which steps of method 900 are performed may vary from one performance to the process of another performance of the process. Steps may also be omitted, combined, renamed, regrouped, be performed on one or more machines, or otherwise depart from the illustrated flow, provided that the process performed is operable and conforms to at least one claim. The steps of FIG. 9 may be performed by the computing device 110 (e.g., via the sensor data processor 114 and/or the deployment processor 116), the system 500 (e.g., via the diffusion feature extractor 512, the background methane estimator 514, the methane compensator 516, the emission leak aggregator 522, the spatial distribution processor 526, and/or the deployment processor 528), or other suitable computing device.

Method 900 begins with step 902. At step 902, reference concentration levels of a pollutant within a geographic region are determined based on environmental characteristics of the geographic region. In some aspects, the reference concentration levels are determined by the pollutant reference module 510. In some embodiments, step 902 includes: determining background methane levels associated with the geographic region; modelling pollutants entering the geographic area from pollutant sources that are outside of the geographic area; and generating the reference concentration levels of the pollutant within the geographic region based on the background methane levels and the modelled pollutants.

At step 904, an estimated emissions probability map for the pollutant is generated based on the reference concentration levels of the pollutant, pollutant source map data, and the environmental characteristics of the geographic region. In some aspects, the estimated emissions probability map for the pollutant corresponds to the emission probability map generated by the emission leak aggregator 522 or the estimated emissions probability map 800. In some embodiments, step 904 includes modelling pollutants entering the geographic area from pollutant sources that are within the geographic area.

At step 906, the geographic region is divided into a plurality of sub-regions based on common dispersion effects. In some aspects, the sub-regions correspond to the sub-regions identified by the spatial distribution processor 526. In some embodiments, dividing the geographic region includes identifying a sub-region as a contiguous cluster of areas that are within the geographic region and have common dispersion effects on the pollutant.

At step 908, a sensor placement map for the geographic region is generated based on the estimated emissions probability map of the pollutant and an estimated dispersion of the pollutant through the plurality of sub-regions, the sensor placement map having locations within the geographic region identified for placement of pollutant sensors. In some aspects, the sensor placement map corresponds to the sensor placement map generated by the deployment processor 528.

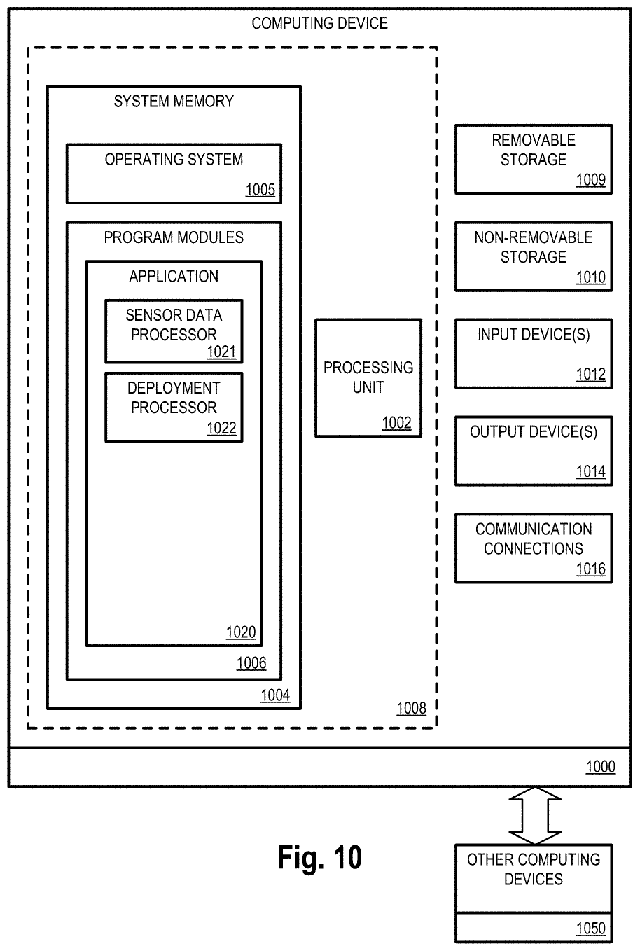

FIGS. 10, 11, and 12 and the associated descriptions provide a discussion of a variety of operating environments in which aspects of the disclosure may be practiced. However, the devices and systems illustrated and discussed with respect to FIGS. 10-12 are for purposes of example and illustration and are not limiting of a vast number of computing device configurations that may be utilized for practicing aspects of the disclosure, as described herein.

FIG. 10 is a block diagram illustrating physical components (e.g., hardware) of a computing device 1000 with which aspects of the disclosure may be practiced. The computing device components described below may have computer executable instructions for implementing a pollutant sensor deployment application 1020 on a computing device (e.g., computing device 110), including computer executable instructions for pollutant sensor deployment application 1020 that can be executed to implement the methods disclosed herein. In a basic configuration, the computing device 1000 may include at least one processing unit 1002 and a system memory 1004. Depending on the configuration and type of computing device, the system memory 1004 may comprise, but is not limited to, volatile storage (e.g., random access memory), non-volatile storage (e.g., read-only memory), flash memory, or any combination of such memories. The system memory 1004 may include an operating system 1005 and one or more program modules 1006 suitable for running pollutant sensor deployment application 1020, such as one or more components with regard to FIG. 1 and, in particular, sensor data processor 1021 (e.g., corresponding to sensor data processor 114), and deployment processor 1022 (e.g., corresponding to deployment processor 116).

The operating system 1005, for example, may be suitable for controlling the operation of the computing device 1000. Furthermore, aspects of the disclosure may be practiced in conjunction with a graphics library, other operating systems, or any other application program and is not limited to any particular application or system. This basic configuration is illustrated in FIG. 10 by those components within a dashed line 1008. The computing device 1000 may have additional features or functionality. For example, the computing device 1000 may also include additional data storage devices (removable and/or non-removable) such as, for example, magnetic disks, optical disks, or tape. Such additional storage is illustrated in FIG. 10 by a removable storage device 1009 and a non-removable storage device 1010.

As stated above, a number of program modules and data files may be stored in the system memory 1004. While executing on the processing unit 1002, the program modules 1006 (e.g., pollutant sensor deployment application 1020) may perform processes including, but not limited to, the aspects, as described herein. Other program modules that may be used in accordance with aspects of the present disclosure, and in particular for determining locations for pollutant sensors, may include sensor data processor 1021 and deployment processor 1022.

Furthermore, aspects of the disclosure may be practiced in an electrical circuit comprising discrete electronic elements, packaged or integrated electronic chips containing logic gates, a circuit utilizing a microprocessor, or on a single chip containing electronic elements or microprocessors. For example, aspects of the disclosure may be practiced via a system-on-a-chip (SOC) where each or many of the components illustrated in FIG. 10 may be integrated onto a single integrated circuit. Such an SOC device may include one or more processing units, graphics units, communications units, system virtualization units and various application functionality all of which are integrated (or “burned”) onto the chip substrate as a single integrated circuit. When operating via an SOC, the functionality, described herein, with respect to the capability of client to switch protocols may be operated via application-specific logic integrated with other components of the computing device 1000 on the single integrated circuit (chip). Aspects of the disclosure may also be practiced using other technologies capable of performing logical operations such as, for example, AND, OR, and NOT, including but not limited to mechanical, optical, fluidic, and quantum technologies. In addition, aspects of the disclosure may be practiced within a general purpose computer or in any other circuits or systems.

The computing device 1000 may also have one or more input device(s) 1012 such as a keyboard, a mouse, a pen, a sound or voice input device, a touch or swipe input device, etc. The output device(s) 1014 such as a display, speakers, a printer, etc. may also be included. The aforementioned devices are examples and others may be used. The computing device 1000 may include one or more communication connections 1016 allowing communications with other computing devices 1050. Examples of suitable communication connections 1016 include, but are not limited to, radio frequency (RF) transmitter, receiver, and/or transceiver circuitry; universal serial bus (USB), parallel, and/or serial ports.

The term computer readable media as used herein may include computer storage media. Computer storage media may include volatile and nonvolatile, removable and non-removable media implemented in any method or technology for storage of information, such as computer readable instructions, data structures, or program modules. The system memory 1004, the removable storage device 1009, and the non-removable storage device 1010 are all computer storage media examples (e.g., memory storage). Computer storage media may include RAM, ROM, electrically erasable read-only memory (EEPROM), flash memory or other memory technology, CD-ROM, digital versatile disks (DVD) or other optical storage, magnetic cassettes, magnetic tape, magnetic disk storage or other magnetic storage devices, or any other article of manufacture which can be used to store information and which can be accessed by the computing device 1000. Any such computer storage media may be part of the computing device 1000. Computer storage media does not include a carrier wave or other propagated or modulated data signal.

Communication media may be embodied by computer readable instructions, data structures, program modules, or other data in a modulated data signal, such as a carrier wave or other transport mechanism, and includes any information delivery media. The term “modulated data signal” may describe a signal that has one or more characteristics set or changed in such a manner as to encode information in the signal. By way of example, and not limitation, communication media may include wired media such as a wired network or direct-wired connection, and wireless media such as acoustic, radio frequency (RF), infrared, and other wireless media.

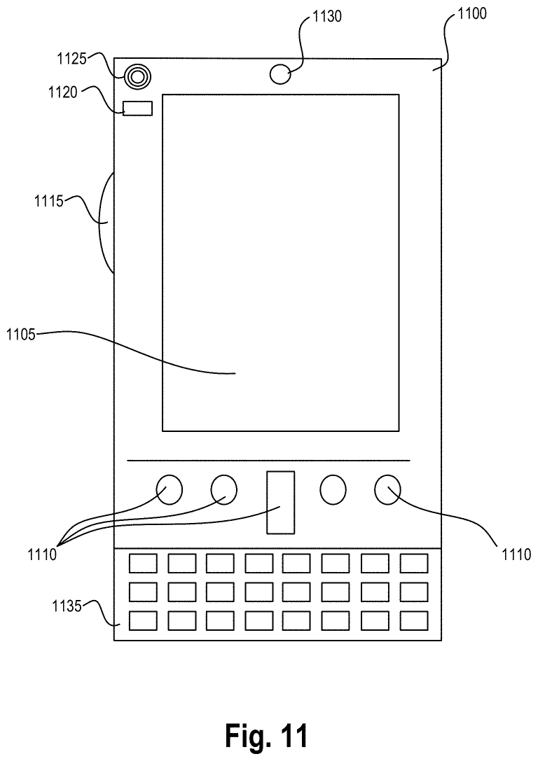

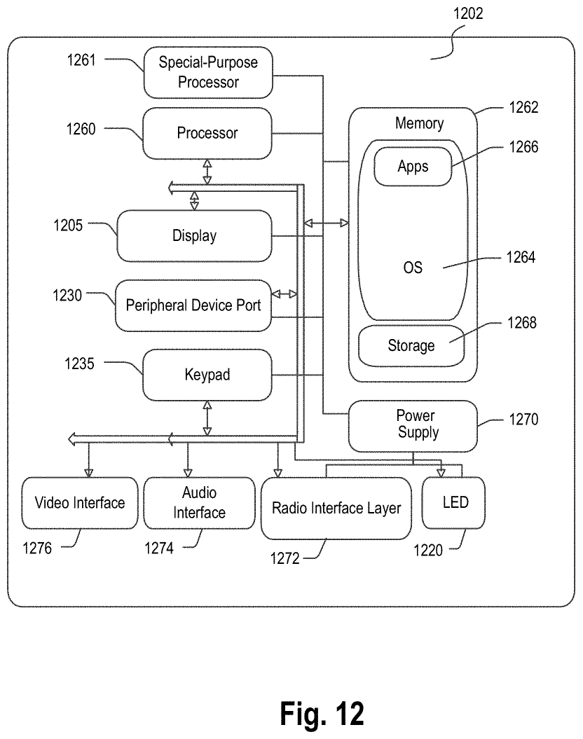

FIGS. 11 and 12 illustrate a mobile computing device 1100, for example, a mobile telephone, a smart phone, wearable computer (such as a smart watch), a tablet computer, a laptop computer, and the like, with which aspects of the disclosure may be practiced. In some aspects, the client may be a mobile computing device. With reference to FIG. 11, one aspect of a mobile computing device 1100 for implementing the aspects is illustrated. In a basic configuration, the mobile computing device 1100 is a handheld computer having both input elements and output elements. The mobile computing device 1100 typically includes a display 1105 and one or more input buttons 1110 that allow the user to enter information into the mobile computing device 1100. The display 1105 of the mobile computing device 1100 may also function as an input device (e.g., a touch screen display). If included, an optional side input element 1115 allows further user input. The side input element 1115 may be a rotary switch, a button, or any other type of manual input element. In alternative aspects, mobile computing device 1100 may incorporate more or less input elements. For example, the display 1105 may not be a touch screen in some aspects. In yet another alternative embodiment, the mobile computing device 1100 is a portable phone system, such as a cellular phone. The mobile computing device 1100 may also include an optional keypad 1135. Optional keypad 1135 may be a physical keypad or a “soft” keypad generated on the touch screen display. In various aspects, the output elements include the display 1105 for showing a graphical user interface (GUI), a visual indicator 1120 (e.g., a light emitting diode), and/or an audio transducer 1125 (e.g., a speaker). In some aspects, the mobile computing device 1100 incorporates a vibration transducer for providing the user with tactile feedback. In yet another aspect, the mobile computing device 1100 incorporates input and/or output ports, such as an audio input (e.g., a microphone jack), an audio output (e.g., a headphone jack), and a video output (e.g., a HDMI port) for sending signals to or receiving signals from an external device.

FIG. 12 is a block diagram illustrating the architecture of one aspect of a mobile computing device. That is, the mobile computing device 1100 can incorporate a system (e.g., an architecture) 1202 to implement some aspects. In one embodiment, the system 1202 is implemented as a “smart phone” capable of running one or more applications (e.g., browser, e-mail, calendaring, contact managers, messaging clients, games, and media clients/players). In some aspects, the system 1202 is integrated as a computing device, such as an integrated personal digital assistant (PDA) and wireless phone.

One or more application programs 1266 may be loaded into the memory 1262 and run on or in association with the operating system 1264. Examples of the application programs include phone dialer programs, e-mail programs, personal information management (PIM) programs, word processing programs, spreadsheet programs, Internet browser programs, messaging programs, and so forth. The system 1202 also includes a non-volatile storage area 1268 within the memory 1262. The non-volatile storage area 1268 may be used to store persistent information that should not be lost if the system 1202 is powered down. The application programs 1266 may use and store information in the non-volatile storage area 1268, such as email or other messages used by an email application, and the like. A synchronization application (not shown) also resides on the system 1202 and is programmed to interact with a corresponding synchronization application resident on a host computer to keep the information stored in the non-volatile storage area 1268 synchronized with corresponding information stored at the host computer.

The system 1202 has a power supply 1270, which may be implemented as one or more batteries. The power supply 1270 may further include an external power source, such as an AC adapter or a powered docking cradle that supplements or recharges the batteries.

The system 1202 may also include a radio interface layer 1272 that performs the function of transmitting and receiving radio frequency communications. The radio interface layer 1272 facilitates wireless connectivity between the system 1202 and the “outside world,” via a communications carrier or service provider. Transmissions to and from the radio interface layer 1272 are conducted under control of the operating system 1264. In other words, communications received by the radio interface layer 1272 may be disseminated to the application programs 1266 via the operating system 1264, and vice versa.

The visual indicator 1220 may be used to provide visual notifications, and/or an audio interface 1274 may be used for producing audible notifications via an audio transducer 1225 (e.g., audio transducer 1225 illustrated in FIG. 12). In the illustrated embodiment, the visual indicator 1220 is a light emitting diode (LED) and the audio transducer 1225 may be a speaker. These devices may be directly coupled to the power supply 1270 so that when activated, they remain on for a duration dictated by the notification mechanism even though the processor 1260 and other components might shut down for conserving battery power. The LED may be programmed to remain on indefinitely until the user takes action to indicate the powered-on status of the device. The audio interface 1274 is used to provide audible signals to and receive audible signals from the user. For example, in addition to being coupled to the audio transducer 1225, the audio interface 1274 may also be coupled to a microphone to receive audible input, such as to facilitate a telephone conversation. In accordance with aspects of the present disclosure, the microphone may also serve as an audio sensor to facilitate control of notifications, as will be described below. The system 1202 may further include a video interface 1276 that enables an operation of peripheral device 1230 (e.g., on-board camera) to record still images, video stream, and the like.

A mobile computing device 1200 implementing the system 1202 may have additional features or functionality. For example, the mobile computing device 1200 may also include additional data storage devices (removable and/or non-removable) such as, magnetic disks, optical disks, or tape. Such additional storage is illustrated in FIG. 12 by the non-volatile storage area 1268.

Data/information generated or captured by the mobile computing device 1200 and stored via the system 1202 may be stored locally on the mobile computing device 1200, as described above, or the data may be stored on any number of storage media that may be accessed by the device via the radio interface layer 1272 or via a wired connection between the mobile computing device 1200 and a separate computing device associated with the mobile computing device 1200, for example, a server computer in a distributed computing network, such as the Internet. As should be appreciated such data/information may be accessed via the mobile computing device 1200 via the radio interface layer 1272 or via a distributed computing network. Similarly, such data/information may be readily transferred between computing devices for storage and use according to well-known data/information transfer and storage means, including electronic mail and collaborative data/information sharing systems.

As should be appreciated, FIGS. 11 and 12 are described for purposes of illustrating the present methods and systems and is not intended to limit the disclosure to a particular sequence of steps or a particular combination of hardware or software components.

The description and illustration of one or more aspects provided in this application are not intended to limit or restrict the scope of the disclosure as claimed in any way. The aspects, examples, and details provided in this application are considered sufficient to convey possession and enable others to make and use the best mode of claimed disclosure. The claimed disclosure should not be construed as being limited to any aspect, example, or detail provided in this application. Regardless of whether shown and described in combination or separately, the various features (both structural and methodological) are intended to be selectively included or omitted to produce an embodiment with a particular set of features. Having been provided with the description and illustration of the present application, one skilled in the art may envision variations, modifications, and alternate aspects falling within the spirit of the broader aspects of the general inventive concept embodied in this application that do not depart from the broader scope of the claimed disclosure.

What is claimed is:

1. A method for pollutant sensor placement, the method comprising:
receiving data about environmental characteristics across a geographic region from a plurality of environmental sensors, wherein the geographic region includes one or more pollutant sources that emit a pollutant;
transforming the received data from one or more of the plurality of environmental sensors into common data having a common grid across the geographic region;
dividing the geographic region into a plurality of sub-regions based on the common data; and
determining locations within the geographic region for placement of pollutant sensors based on estimated dispersion of the pollutant through the plurality of sub-regions.

2. The method of claim 1, wherein dividing the geographic region into the plurality of sub-regions comprises identifying a sub-region as a contiguous cluster of areas that are within the geographic region and have common dispersion effects on the pollutant.

3. The method of claim 2, wherein transforming the received data comprises interpolating first data about the environmental characteristics from a first data source to generate second data that is aligned with the common grid, wherein the common grid is associated with a second data source.

4. The method of claim 3, wherein interpolating the first data is one or both of a temporal interpolation and a spatial interpolation.

5. The method of claim 3, wherein the first data source and the second data source are selected from a land-use data source, a meteorological data source, a pollutant facility data source, and a satellite-based pollutant emissions data source.

6. The method of claim 2, wherein each of the plurality of sub-regions belongs to a sub-region group of a plurality of sub-region groups, each sub-region within a sub-region group having common dispersion effects on the pollutant.

7. The method of claim 6, wherein at least some sub-regions within a same sub-region group are non-contiguous.

8. The method of claim 2, wherein the identified sub-region has the common dispersion effects on the pollutant over different seasons.

9. The method of claim 2, wherein determining the locations within the geographic region for placement of the pollutant sensors comprises determining a number of the pollutant sensors and the locations of the pollutant sensors that provide a minimum pollutant detectability threshold.

10. The method of claim 9, wherein determining the locations within the geographic region for placement of the pollutant sensors further comprises selecting the pollutant sensors from a plurality of pollutant sensors having different pollutant detection sensitivities.

11. A method for pollutant sensor placement comprising:
determining reference concentration levels of a pollutant within a geographic region based on environmental characteristics of the geographic region;
generating an estimated emissions probability map for the pollutant based on the reference concentration levels of the pollutant, pollutant source map data, and the environmental characteristics of the geographic region;
dividing the geographic region into a plurality of sub-regions based on common dispersion effects;
generating a sensor placement map for the geographic region based on the estimated emissions probability map of the pollutant and an estimated dispersion of the pollutant through the plurality of sub-regions, the sensor placement map having locations within the geographic region identified for placement of pollutant sensors.

12. The method of claim 11, wherein determining the reference concentration levels of the pollutant comprises:
determining background methane levels associated with the geographic region;
modelling pollutants entering the geographic area from pollutant sources that are outside of the geographic area; and
generating the reference concentration levels of the pollutant within the geographic region based on the background methane levels and the modelled pollutants.

13. The method of claim 11, wherein dividing the geographic region into the plurality of sub-regions comprises identifying a sub-region as a contiguous cluster of areas that are within the geographic region and have common dispersion effects on the pollutant.

14. The method of claim 11, wherein generating the estimated emissions probability map for the pollutant comprises modelling pollutants entering the geographic area from pollutant sources that are within the geographic area.

15. A system for pollutant sensor placement, the system comprising:
a staging database configured to receive data about environmental characteristics across a geographic region from a plurality of environmental sensors, wherein the geographic region includes one or more pollutant sources that emit a pollutant;
a sensor data processor configured to transform the received data from one or more of the plurality of environmental sensors into common data having a common grid across the geographic region; and
a deployment processor configured to:
divide the geographic region into a plurality of sub-regions based on the common data; and
determine locations within the geographic region for placement of pollutant sensors based on estimated dispersion of the pollutant through the plurality of sub-regions.

16. The system of claim 15, wherein the deployment processor is further configured to identify a sub-region as a contiguous cluster of areas that are within the geographic region and have common dispersion effects on the pollutant, wherein each of the plurality of sub-regions belongs to a sub-region group of a plurality of sub-region groups, each sub-region within a sub-region group having common dispersion effects on the pollutant.

17. The system of claim 16, wherein at least some sub-regions within a same sub-region group are non-contiguous.

18. The system of claim 15, wherein the first data source and the second data source are selected from a land-use data source, a meteorological data source, a pollutant facility data source, and a satellite-based pollutant emissions data source.

19. The system of claim 15, wherein the deployment processor is configured to determine a number of the pollutant sensors and the locations of the pollutant sensors that provide a minimum pollutant detectability threshold.

20. The system of claim 15, wherein the deployment processor is configured to select the pollutant sensors from a plurality of pollutant sensors having different pollutant detection sensitivities.
